(19) United States
(12) Patent Application Publication
(10) Pub. No.: US 2023/0256263 A1
(43) Pub. Date: Aug. 17, 2023

(54) Hafnium-Based Gamma Radiography Sources, Gamma Radiation Exposure Devices, and Methods of Gamma Radiography

(71) Applicant: QSA Global Inc., Burlington, MA (US)

(72) Inventor: Mark Shilton, Chelmsford, MA (US)

(21) Appl. No.: 18/163,787

(22) Filed: Feb. 2, 2023

Related U.S. Application Data
(60) Provisional application No. 63/309,297, filed on Feb. 11, 2022.

(51) Int. Cl. A61N 5/10

(52) U.S. Cl. CPC A61N 5/1001; A61N 2005/1094; A61N 2005/1024

(57) ABSTRACT

Disclosed example gamma radiography sources include an encapsulated quantity of at least 10 Ci of Hafnium-175 (Hf-175). Disclosed example gamma radiation exposure devices include: a gamma radiation source comprising a quantity of Hf-175; and a shielding device configured to attenuate gamma radiation emitted by the gamma radiation source while the gamma radiation source is in a stored position, and configured to allow the gamma radiation source to be moved to an exposed position for gamma radiation exposure.

### FIELD OF THE DISCLOSURE

This disclosure relates generally to gamma radiography and, more particularly, to hafnium-based gamma radiography sources, gamma radiation exposure devices, and methods of gamma radiography.

### BACKGROUND

Industrial radiography is often used for producing images of objects that are otherwise difficult to inspect, and involves exposing a source of high-energy radiation (e.g., gamma rays) and collecting penetrating and/or scattered rays to form a radiographic image. When not in use, gamma ray sources, such as radioactive isotopes, are stored in shielding devices.

### SUMMARY

Hafnium-based gamma radiography sources, gamma radiation exposure devices, and methods of gamma radiography are disclosed, substantially as illustrated by and described in connection with at least one of the figures, as set forth more completely in the claims.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIGS. 1A and 1B illustrate an example radiography system having a source exposure device for providing radiation for radiography, in accordance with aspects of this disclosure.

FIGS. 2A and 2B illustrate another example radiography system having a source projector device for providing radiation for radiography, in accordance with aspects of this disclosure.

FIG. 3 is a cross-sectional view of an example encapsulated hafnium-based gamma radiation source, in accordance with aspects of this disclosure.

FIG. 4 is a more detailed view of the example hafnium-based gamma radiation source of FIG. 3.

FIG. 5 is a flowchart representative of an example method which may be performed to manufacture a radiography source and radiography system.

FIG. 6 is a flowchart representative of an example method which may be performed to control exposure of a radiography source using the system of FIGS. 1A and 1B or 2A and 2B.

The figures are not necessarily to scale. Wherever appropriate, similar or identical reference numerals are used to refer to similar or identical components.

### DETAILED DESCRIPTION

For the purpose of promoting an understanding of the principles of the claimed technology and presenting its currently understood, best mode of operation, reference will be now made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would typically occur to one skilled in the art to which the claimed technology relates.

Conventional isotope gamma radiation sources have used radionuclides including Co-60, Ir-192, Se-75, and Yb-169, each with respective half-lives, specific activities, and mean emission energy, which are variably useful for different radiographic applications. For example, different radionuclides may be selected depending on the target material type(s) and/or thickness(es). The radionuclides Co-60, Ir-192, Se-75, and Yb-169 have been shown to be commercially useful for radiography due to their emissive qualities and manufacturability.

Hafnium is a ductile, silver-grey metal. Hafnium has high resistance to chemical attack and melts at 2233° C. (compare iridium at 2466° C.). Hf-175 is a radioisotope with a 70 day half-life, and emits a prominent 343 keV gamma ray with 87% abundance. The emission energy of Hf-175 is between those of Ir-192 (mean of 370 keV) and Se-75 (mean of 215 keV), and the half-life is similar to Ir-192 (74 days). Hf-175 has several beneficial nuclear characteristics, which make Hf-175 attractive as a gamma radiography nuclide. The target nuclide needed to make Hf-175 is isotopically enriched, stable Hf-174, which has a very high activation cross section and low burn-away cross section, but low natural isotopic abundance.

The principal gamma ray emission energies of Hf-175 are listed below in Table 1. There is one high-abundance emission at 343 keV (87%), with several other low abundance emissions between 89.4 keV and 432.8 keV. These energies preferably match the gamma ray attenuation characteristics of many common fixtures found in industry that have joints, such as pipelines, flanges, and tanks and their weldments. The practical working thickness range in copper, nickel, or steel alloys for Hf-175 is estimated to be 8-45 mm, but may be variable depending on the sensitivity requirement and imaging methodology used.

TABLE 1
Hf-175 Emission Energies and Abundance

| Energy | Gamma Emission Abundance |
|---|---|
| 89.4 keV | 2.40 photons per 100 decays |
| 113.8 keV | 0.29 photons per 100 decays |
| 143.9 keV | 0.01 photons per 100 decays |
| 161.3 keV | 0.02 photons per 100 decays |
| 229.6 keV | 0.77 photons per 100 decays |
| 319.0 keV | 0.17 photons per 100 decays |
| 343.4 keV | 87.0 photons per 100 decays |
| 353.6 keV | 0.23 photons per 100 decays |
| 432.8 keV | 1.57 photons per 100 decays |

The target material to produce Hf-175 is Hf-174. Hf-174 is a stable natural isotope, but it has a very low natural isotopic abundance of only 0.16%. This compares with Yb-168 (0.13%), Se-74 (0.89%) and Ir-191 (37.3%). The low natural abundance of Hf-174 would not enable the production of useful Hf-175 gamma radiography sources using natural hafnium.

Hf-175 can be produced by thermal neutron activation of stable Hf-174. Hf-174 has a very high thermal and epithermal neutron capture cross-section, and the resulting Hf-175 has a low burn-away cross-section, both of which improve the manufacturing yield of Hf-175 by thermal and epithermal neutron irradiation of Hf-174. Thermal and epithermal neutron irradiation may be achieved in a nuclear reactor, and is preferably performed at locations in the reactor at which the incidence of fast neutrons (>5 MeV) is limited or minimized to the extent practicable.

In disclosed examples, Hf-174 source material is constructed into thin disks or other shapes to improve (e.g., maximize) the surface area exposed to irradiation. Due to a very high affinity for neutrons, only the first few fractions of millimeter below the surface of an Hf-174 substrate can activate efficiently. Further beneath the surface, the neutron flux will become depleted, making activation inefficient. Activation of Hf-174 by fast neutrons (e.g., neutrons having >5 MeV energy) can produce a small amount of Lu-174m impurity via the Hf-174(n,p)Lu-174m route. Lu-174m emits 1246 keV gamma rays with 6.5% abundance with a half-life of 3.31 years. If present in gamma radiography sources, this could adversely affect the dose rate on shields. Between 5-10 MeV neutron energies, the activation cross section of Hf-174 is extremely low (e.g., <1 mbarn). Between 10-14 MeV neutron energies, the activation cross section increases to 100 mB, but there are very few neutrons with such high energy. Disclosed examples perform activations in a region of the reactor core, that has an increased (e.g., maximized) ratio of combined thermal and epithermal flux relative to the fast neutron flux (e.g., >5 MeV). Such positions and ratios may vary from one reactor to another.

Hf-174 has a very high cross section for epithermal resonance neutron capture (absorption) in the energy range 1 to 1000 eV (10⁻⁶ to 10⁻³ MeV). For reference thermal neutrons have approximate energy of 0.025 eV (2.5×10⁻⁸ MeV). Epithermal neutrons have energies above thermal that range from 0.025 eV up to about 0.4 eV. Some estimations cite the epithermal resonance integral for Hf-174 to be ˜7200 barns relative to the thermal neutron capture cross section of ˜200 barns. In some reactors the epithermal neutron flux can be up to about two-thirds of the thermal neutron flux in some activation positions. In other reactors the epithermal flux may be as low as one twentieth of the thermal flux in some activation positions. The efficiency of Hf-174 activation is expected to be enhanced by irradiating Hf-174 in a type of reactor, and in a position, which has high epithermal neutron flux and also high combined thermal-epithermal flux. For example, it is advantageous for the percentage of epithermal neutron flux to be at least 20% of the thermal neutron flux. It is further advantageous for the percentage of epithermal neutron flux to be at least 30% of the thermal neutron flux, and even further advantageous for the percentage of epithermal neutron flux to be at least 50% of the thermal neutron flux.

Beneficially, Hf-175 has no high-energy gamma rays above 432.8 keV. Therefore, unlike Ir-192, Hf-175 does not produce emissions that can adversely impact surface dose rate of radiological safety shields (e.g., shipping containers, exposure devices). The absence of high energy gamma rays could enable Hf-175 to be shipped and used in a Type A package under the International Atomic Energy Agency (IAEA) Safety Standards, Regulations for the Safe Transport of Radioactive Material, 2012 Edition. Type-A packages are typically used with Se-75 gamma sources, but may be used with Hf-175 gamma sources, despite having a higher average emission energy than Se-75 and a wider useful working range. Additionally, higher activity Hf-175 sources, can be used in heavier-shielded type-B radiography packages (e.g., radiography packages used with Ir-192 sources). Such higher-activity Hf-175 sources allow for higher source output and shorter radiography exposure time.

The focal spot size of a Hf-175 disk stack is determined at least in part on activation and geometric parameters. Hf-174 can be irradiated as discrete unencapsulated disks or pellets (e.g., metal, alloy, Hf-174-containing compound, ceramic-metal composite (cermet), oxide, ceramic, and/or other refractory form(s)), which is advantageous compared with requiring encapsulation prior to irradiation, as is necessary for irradiations of enriched Se-74 to make Se-75. As a result, Hf-175 gamma radiation sources may be designed for particular source activity and/or focal spot size by, for example, varying irradiated disk stack height and/or diameter in the source.

Hf-175 has fewer gamma ray emissions per decay than Se-75; however, because its average output energy is higher than Se-75, Hf-175 has almost the same specific gamma ray constant. The specific gamma ray constant of Hf-175 is 0.204 R/h/Ci at 1 meter, while the specific gamma ray constant of Se-75 is 0.201 R/h/Ci at 1 meter, and the specific gamma ray constant of Ir-192 is 0.46 R/h/Ci at 1 meter.

Other naturally occurring stable isotopes of Hafnium include Hf-176 (5.26%), Hf-177 (18.6%), Hf-178 (27.3%), Hf-179 (13.6%), and Hf-180 (35.1%). Each of these isotopes have high neutron activation cross sections. The presence of any of these isotopes in enriched Hf-174 would depress the neutron flux during activation. Disclosed examples use Hafnium-174 which has been enriched to at least 20%. More preferably, some disclosed examples use Hafnium-174 which has been enriched to at least 50%. Even more preferably some disclosed examples use Hafnium-174 which has been enriched to at least 80%.

Any Hf-180 remaining in enriched Hf-174-containing Hafnium would activate during irradiation to produce radioactive Hf-181. Hf-181 is a radioactive isotope with a 42.4 day half-life. Hf-181 has gamma emissions of 133 keV (36%), 136 keV (6%), 346 keV (15%), 482 keV (80%), 615 keV (0.3%), which are complementary to those of Hf-175 in that the emissions would not adversely interfere with the emissions of Hf-175 for radiographic, storage, and/or transport purposes. However, Hf-181, once produced in the reactor, can further activate to Hf-182, which then decays to Ta-182 or Hf-181 can decay in the reactor to form stable Ta-181 and then Ta-181 can further activate to Ta-182. Ta-182 is a radioisotope impurity with 114.4 day half-life, and which emits high-abundance, high-energy gamma rays which may be undesirable for radiography applications. Therefore, it is desirable to reduce (e.g., minimize) the percentage of Hf-180 in Hf-174 target material, to thereby reduce (e.g., minimize) the amount of Ta-182 impurity that can occur in the final irradiated product. The percentage of Hf-180 in the Hf-174 enriched Hafnium is advantageously <5%, more advantageously <2%, and even more advantageously <0.5%.

Hafnium also forms a stable oxide (HfO2), which is similar to zirconium oxide (ZrO2) and has a high melting point of 2758° C. Hafnium forms a refractory carbide (HfC) with melting point 3305° C., forms a refractory nitride (HfN) with melting point 3310° C., and forms binary and tertiary alloys with non-activating metals such as aluminum (HfAl). These carbides, nitrides, and alloys are alternative chemical and physical forms of hafnium that could be used in sources.

In a gamma radiography source, Hf-175 has a useful working thickness estimated to be between 8 mm and 45 mm. A Hf-175 source having a 100 Ci output is expected to contain about 130 Ci (depending on focal dimension). For Hf-175 radiography sources having less than the 3.0 TBq (81.08 Ci) Type-A limit for both normal form and special form, a Type A package may be used for transport and exposure.

In disclosed examples, after activation, the Hf-175 may be formed using any combination of stacked disks, microspheres, pressed pellets, coiled wires, annuli, profiled disks, and/or any other desired shape. For example, Hf-175 sources could be constructed to contain stacked metal disks, compressed hafnium metal pellets, stacked hafnium oxide pellets, stacked hafnium nitride pellets, stacked hafnium carbide pellets, and/or stacked hafnium aluminide pellets, and/or microspheres thereof, or as a cermet in combination with non-activating supporting materials.

The activation yield of Hf-175 determines the achievable focal spot size and activity of radiography sources, and depends on the Hf-174 enrichment, the reactor neutron flux, and the irradiation time. The focal dimension of Hf-175 sources depends on the activation yield, the film sensitivity, and contrast achieved by the 343 keV gamma rays. Smaller focal dimensions compared with than Ir-192 sources may be achievable, and/or higher contrast and resolution, as a result of lower mean emission energies in Hf-175 and the absence of the high energy gamma rays that are present in Ir-192 which can fog film and digital plates used in gamma radiography procedures (sometimes referred to as high noise in Ir-192 images).

Mass attenuation coefficients for Hf-175 are intermediate between Ir-192 and Se-75. However, the MVL (1000th layer attenuation) for Hf-175 is less than Se-75. As a result, although Hf-175 has a higher average emission energy than Se-75 and higher HVL than Se-75, Hf-175 can be used in radiation shields meeting the same requirements as Se-75 sources (assuming appreciable high energy gamma ray impurities such as Lu-174m or Ta-182 are not present).

Disclosed example methods involve isotopic enrichment of the target Hf-174 nuclide to economically manufacture Hf-175 radiography sources. Example methods of isotopic enrichment include electromagnetic enrichment, laser excitation enrichment, thermal diffusion enrichment, chromatographic enrichment, or gas centrifuge enrichment. Hafnium-174 may be most economically enriched via gas centrifugation with a compound of Hafnium-174 which has sufficiently high, low-temperature vapor pressure and sufficient chemical and physical stability at high-temperature, such as hafnium borohydride Hf(BH₄)₄ or one of its analogues. One or more hydrogen atoms in BH₄ can potentially be substituted by alternate ligands to increase physical stability of the molecule, while maintaining or increasing the vapor pressure. Boron has two stable isotopes, Boron-10 and Boron-11. In some examples, a hafnium borohydride complex would contain a single isotope of highly enriched Boron (e.g., highly enriched B-10, highly-enriched B-11) to increase enrichment efficiency.

In other examples, Hafnium can also be enriched by atomic vapor laser isotope enrichment (AVLIS) or by molecular laser isotope enrichment (MLIS) using elemental hafnium or other molecules, which have suitable physical and chemical properties to enable excitation of the Hf-174 isotope in preference to other hafnium isotopes, by a highly tuned laser beam.

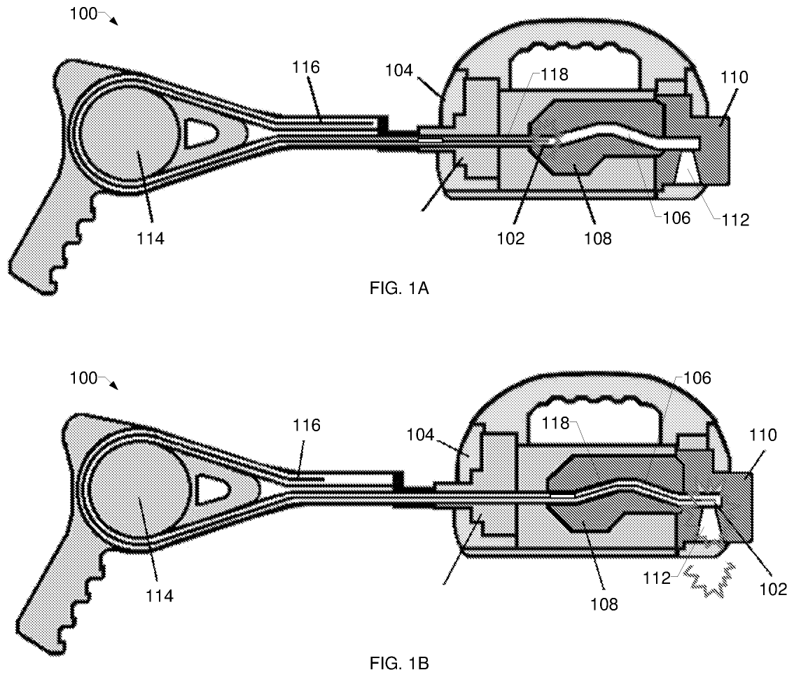

FIGS. 1A and 1B illustrate an example radiography system 100 having a source exposure device for emitting radiation to perform radiography. The radiography system 100 includes a radiography source 102 (also referred to herein as a radiation source) which is contained within a radiography source housing 104 (also referred to as a package). The example housing 104 is configured to meet the requirements of a Type A package under the International Atomic Energy Agency Safety Standards, Regulations for the Safe Transport of Radioactive Material, 2012 Edition.

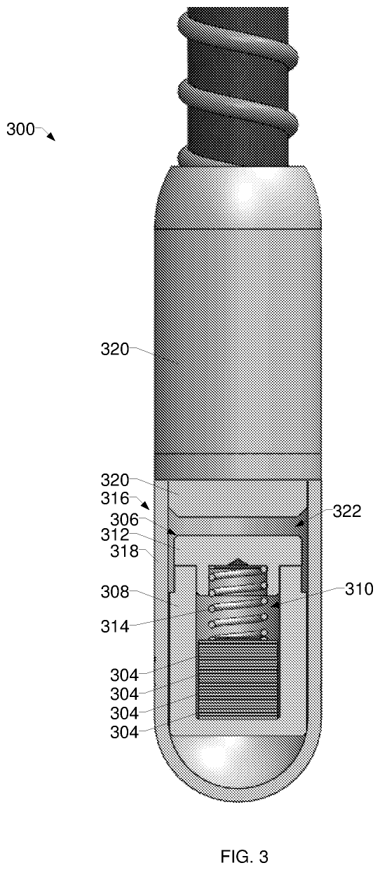

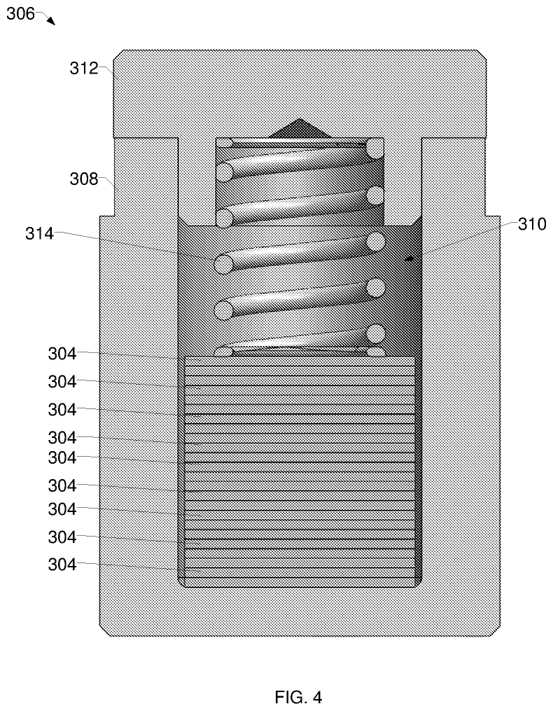

The example radiography source 102 is a quantity of Hafnium-175, or thermal neutron-irradiated Hafnium-174, which emits radiation (e.g., gamma rays) due to decay of the material. Upon irradiation by thermal neutrons in a reactor, at least a portion of Hf-174-containing Hafnium is activated to Hf-175, while a portion may remain as Hf-174. Therefore, as used herein, neutron-irradiated, enriched Hf-174 may be understood to include a combination of Hf-175 and Hf-174. For the purposes of gamma radiography, higher yields of Hf-175 are advantageous. An example implementation of the radiography source 102 is disclosed below with reference to FIGS. 3 and 4.

The radiography source 102 is positioned in a source tube 106 within a shield 108 within the housing 104. The example shield 108 is a tungsten-based shield. When positioned in a stored position as shown in FIG. 1A, emissions from the radiography source 102 are shielded by the shield 108. As an end of the shield 108, the source tube 106 and shield are connected to an outlet port 110 or collimator. The outlet port 110 may be a similar shielding material as the shield 108, but includes an aperture 112 through which gamma ray emissions may be projected from the radiography system 100 in a desired direction.

To project gamma rays for radiography, the example radiography source 102 is moved via the source tube 106 into a projection position within the outlet port 110 and adjacent the aperture 112, as illustrated in FIG. 1B. For example, a remote control device 114 may be removably attached to the radiography source 102 to control a position of the radiography source 102 (e.g., the stored position, the projection position). For example, the remote control 114 may physically engage the control cable 116 to advance or retract the control cable 116 relative to the remote control 114. By connecting the remote control device 114 to the source 102 via a control cable 116, the remote control device 114 may be manipulated (e.g., cranked) to push or pull the radiography source 102 to a desired position within the housing 104.

To control the position of the radiography source 102, the radiography source housing 104 enables connection of the control cable 116 to the radiography source 102 for exposure and retraction of the radiography source 102. The control cable 116 may be physically attached or connected to a source wire (also referred to as a pigtail) connector 118 that is physically coupled to the radiography source 102.

When engaged, the control cable 116 is controlled to extend into and through the source tube 106 to push the radiography source 102 to an exposed position adjacent the aperture 112. Conversely, the control cable 116 is retracted to pull the radiography source 102 from the exposed position back into the source tube 106 to the shielded position, at which time the control cable 116 may be detached from the radiography source 102.

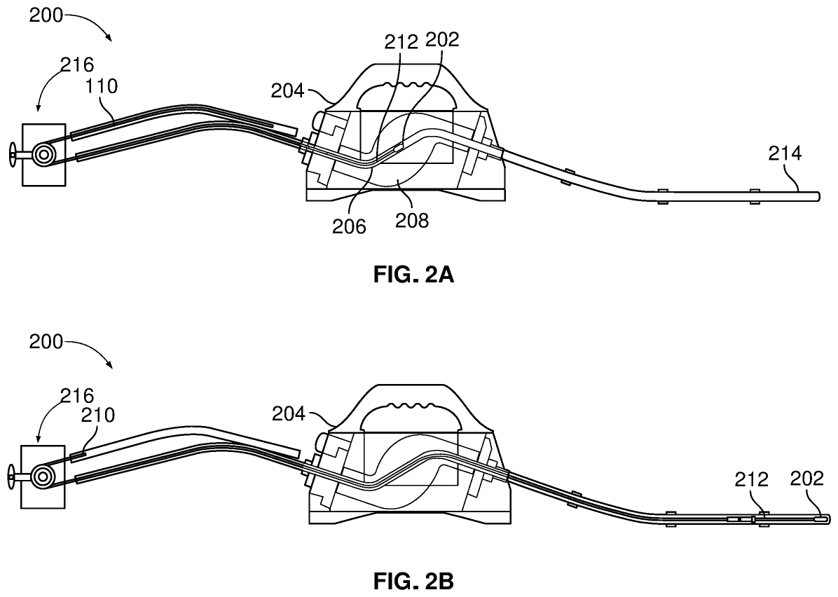

FIGS. 2A and 2B illustrate example radiographic system 200 for providing radiation for radiography. The radiographic system 200 of FIG. 2 includes a radiography source 202 which is contained within a radiography source housing 204. Like the radiography source 102, the example radiography source 202 is a quantity of Hafnium-175, which emits radiation (e.g., gamma rays) due to decay of the material. In contrast with the example projector of FIGS. 1A and 1B, the example system 200 of FIGS. 2A and 2B extend the source 202 to a position external to the housing 204 for exposing the source 202, which may require larger exclusion zones during radiography than the example system 100 of FIGS. 1A and 1B.

The radiography source housing 204 includes an S-shaped source tube 206 within a shield 208. The source tube 206 provides a pathway for the radiography source 202 to be exposed to an exterior of the shield 208, which in this example is constructed from depleted uranium, and retracted to a shielded position within the interior of the shield 208. FIG. 2A illustrates the radiography source 202 in the shielded position, and FIG. 2B illustrates the radiography source 202 in an exposed position. The position of the radiography source 202 may be controlled via a control cable 210 and remote control 216 similar to the control cable 116 and the remote control 120 of FIGS. 1A and 1B.

In the system 200 of FIGS. 2A and 2B, the exposed position of the radiography source 202 may be controlled by a guide tube 214, through which the radiography source 202 travels as the source 202 is pushed by the control cable 210. The control cable 210 has sufficient column strength to push the radiography source 202 through the source tube 206 and through the guide tube 214.

FIG. 3 is a cross-sectional view of an example encapsulated hafnium-based gamma radiation source 300. The example source 300 of FIG. 3 may be used to implement the radiography sources 102, 202 of FIGS. 1A-2B. The source 300 includes a quantity of Hafnium-175 302.

In the example of FIGS. 3 and 4, the Hafnium-175 is formed as a stack of disks 304. The disks 304 may include metallic Hf-175, an alloy of Hf-175 (e.g., containing other metals, such as aluminum, which do not activate to produce gamma emissions which adversely impact the application), a refractory material including Hf-175 (e.g., an oxide of Hf-175 or other material that is resistant to degradation, decomposition, and/or loss of strength at high temperature and does not activate to produce gamma emissions, which adversely impact the application), a composite material including Hf-175, a cermet including Hf-175, and/or any other compound or material containing a sufficient amount of Hf-175. Example compounds containing Hf-175 may include borides, carbides, nitrides, selenides (including enriched Se-74), and/or silicides. In some examples, either intentionally or due to impurities, the disks may further contain one or more activated materials which emit complementary gamma emissions (e.g., non-interfering emission) to the Hf-175 energies (e.g., Hf-181, Se-75).

The stack of disks 304 may be selected to provide a desired quantity of gamma radiation emissions (e.g., based on the radiography application), and the disks 304 are compressed to a desired stack height to provide the desired focal dimension. In some examples, the stack of disks 304 is sized to generate at least 10 Ci of gamma radiation. In some such examples, the stack of disks 304 is sized to generate between 10-81 Ci of gamma radiation or, in some examples, between 40-81 Ci of gamma radiation (e.g., if the source is used in a Type-A radiography package). If a Type-B radiography package is used (e.g., a package typically used for Ir-192 sources), disks could be sized and stacked to produce higher source activities, potentially up to 500 Ci.

The example disks 304 may be flat, curved, or have any other shape and/or profile, which may include deformations, cutouts, and/or any other internal or circumferential features to improve irradiation yield, specific activity, and/or focal dimension. However, in other examples the disks 304 may be replaced and/or supplemented with microspheres, pressed pellets, coiled wires, annuli, profiled disks, and/or any other shape. The disks 304, or other shape, may include grooves and/or curvature, and/or different disks 304 in the stack may have different shapes prior to compression and encapsulation to control the stack density and/or stack profile upon compression and/or encapsulation. Examples of stacked disks and profiled disks which may be used to form the disks 304 are disclosed in U.S. Pat. No. 11,116,992, granted Sep. 14, 2021, entitled “Gamma radiation source comprising low-density deformable/compressible iridium alloy and an encapsulation.” The entirety of U.S. Pat. No. 11,116,992 is incorporated herein by reference.

The disks 304 of FIG. 3 are encapsulated in multiple layers of encapsulation, and attached to a pigtail for physical control of the position of the radiation source 300 within a housing. FIG. 4 is a more detailed view of the example hafnium-based gamma radiation source of FIG. 3 including a first encapsulation 306. The example first encapsulation of FIGS. 3 and 4 is titanium capsule having multiple pieces. A first piece 308 includes a cavity 310 into which the disks 304 are inserted. A second piece 312 (e.g., a cover or cap) is attached to the first piece 308 to secure the disks 304 within the first encapsulation 306. For example, the second piece 312 may be welded, threaded, press fit, and/or any other method of attachment the first piece 308. In other examples, the first encapsulation 306 may be deformed, such as by crimping the first piece 308 and/or the second piece 312, or via another physical deformation process. As shown in FIG. 3, the first encapsulation 306 may include a spring 314 or other biasing element between the pieces 308, 312 to bias the stack of disks 304 towards a desired shape and/or position, and/or to reduce or elimination shifting of the disks 304 that could affect the emission profile.

The first encapsulation 306 containing the disks 304 is encapsulated by a second encapsulation 316, which may be constructed using titanium or other rugged, non-shielding encapsulation material. Similar to the first encapsulation 306, the second encapsulation 316 includes a first piece 318 and a second piece 320. The first piece 318 includes a cavity 322 into which the first encapsulation 306 is inserted, and the cavity 322 is then closed or sealed via attachment of the second piece 320 to the first piece 318. The second encapsulation 316 may have an external profile and/or other features to, for example, enable exertion of increased force on the disks 304 (via the first encapsulation), control the movement and/or rotation of the source 300 within a source tube, and/or attach a pigtail to the source 300 for attachment of a control cable.

In the example of FIG. 3, the second piece 320 of the second encapsulation 316 is connected to a control cable 324, which may implement the control cables 116 or 210 of FIGS. 1A-2B.

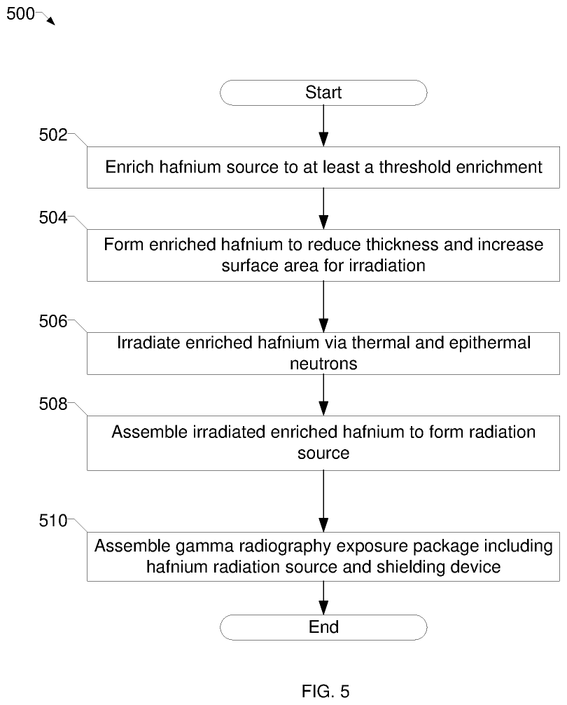

FIG. 5 is a flowchart representative of an example method 500 which may be performed to manufacture a radiography source and radiography system.

At block 502, a quantity of Hf-174 is enriched to at least a threshold enrichment. For example, the enrichment may be performed on elemental Hf-174, a sufficiently stable hafnium molecule that can be vaporized at the temperature required for enrichment without decomposing (e.g., hafnium borohydride (Hf(BH₄)₄, hafnium tetra-fluoride HfF₄), and/or any other appropriate compound for the selected enrichment technique. Enrichment may be performed using electromagnetic enrichment, laser excitation enrichment, thermal diffusion enrichment, chromatographic enrichment, gas centrifuge enrichment, and/or any other desired enrichment technique, which may depend on the available compound of Hf-174. In some examples, the enrichment involves producing Hafnium-174-enriched Hafnium by performing atomic vapor laser isotope separation (AVLIS) of elemental Hafnium. In some other examples, the enrichment involves performing molecular laser isotope separation (MLIS) of a compound containing Hafnium-174. The enrichment threshold may be 20%, 50%, 80%, and/or any other or achievable level of enrichment, while the percentage of Hf-180 in Hf-174 should be reduced (e.g., minimized) to the extent practical.

At block 504, the enriched Hf-174 is formed to reduce the thickness and increase the surface area for irradiation. For example, the enriched Hf-174 may be formed as flat, angled, curved, and/or profiled disks to increase the surface area for irradiation. Because the affinity for neutrons is very high in Hf-174, increasing the surface area and reducing thickness for a given quantity of Hf-174 increases the yield of Hf-175 during irradiation.

At block 506, the formed, enriched Hf-174 is irradiated via thermal and epithermal neutrons. For example, while the incidence of higher-energy neutrons >5 MeV may not be eliminated, the formed, enriched Hf-174 may be placed in location of a nuclear reactor in which the ratio of combined thermal and epithermal neutrons to higher-energy neutrons (e.g., fast neutrons >5 MeV) is maximized or as high as practicable.

At block 508, the irradiated, enriched Hf-174 is assembled to form a radiation source (e.g., the radiation source 300 of FIG. 3). For example, a set of disks 304 may be stacked, compressed, and inserted into one or more encapsulation layers (e.g., first encapsulation 306 and second encapsulation 316 of FIGS. 3 and 4). Other assembly methods and/or steps may be used, depending on the form of the irradiated, enriched Hf-174 (e.g., stacked disks, microspheres, pressed pellets, coiled wires, annuli, profiled disks, etc.).

At block 510, a gamma radiography exposure package is assembled, including the Hf-175 radiation source and a shielding device. For example, the source 300 may be assembled into the radiography system 100 of FIGS. 1A and 1B as the radiography source 102 and/or into the radiography system 200 of FIGS. 2A and 2B as the radiography source 102.

Upon assembly of the gamma radiography exposure package, the example method 500 then ends.

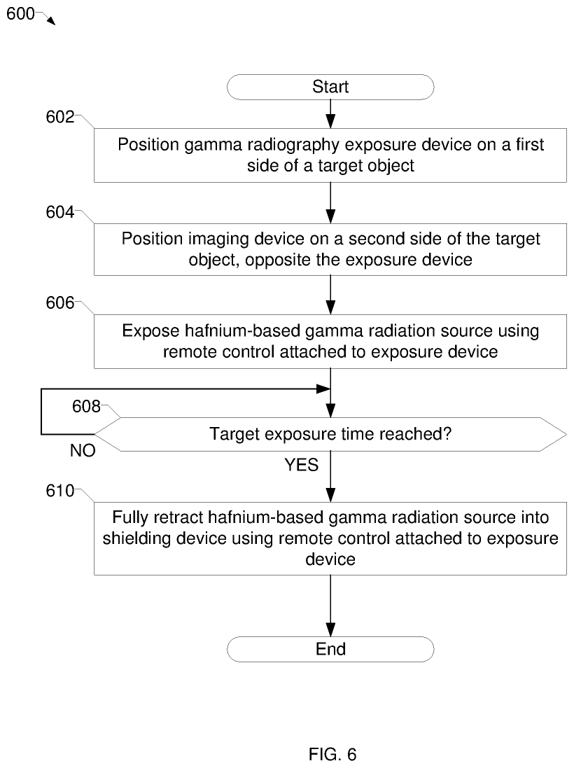

FIG. 6 is a flowchart representative of an example method 600 which may be performed to control exposure of a radiography source using the system of FIGS. 1A and 1B or 2A and 2B. The example method 600 is described below with reference to the radiography system 100 of FIGS. 1A and 1B.

At block 602, the gamma radiography exposure device (e.g., the radiography system 100) is positioned on a first side of a target object (e.g., an object to be radiographed). The radiography system 100 containing Hf-175 as the radiographic element may be selected for particular thicknesses and/or types of materials to be scanned.

At block 604, an imaging device (e.g., radiographic film, a digital radiograph, etc.) is positioned on a second side of the target object, opposite the radiography system 100 as the exposure device. In some examples, such as in an exposure device having a collimator or other directional output of the radiation, the exposure device is oriented to output the gamma radiation toward the imaging device through the target object.

At block 606, the Hf-175 gamma radiation source 102 is exposed using a remote control (e.g., the remote control 120) attached to the source 102. For example, the gamma radiation source 102 may be physically moved from a shielded position (e.g., illustrated in FIG. 1A) to an exposed position (e.g., illustrated in FIG. 1B) by controlling the control cable 116 via the remote control 120.

The Hf-175 gamma radiation source 102 is left in the exposed position for a target time to obtain the desired radiograph. At block 608, if the target exposure time is not reached, the source 102 is left in the exposed position. When the target exposure time is reached (block 608), at block 610 the gamma radiation source 102 is fully retracted into the housing 104 and the shield 108 to the shielded position. The example method 600 may then end.

Relative to conventional, Selenium-75-based radiography sources and/or Iridium-192-based radiography sources, Hf-175-based radiography sources have favorable emission energies (e.g., a prominent 343 keV gamma ray, and absence of high energy gamma rays) which enable the production of high contrast, high resolution images with reduced or eliminated fogging. Hf-175 sources have comparable detection efficiency and comparable image quality to Se-75, and the same light-weight, Type-A exposure devices can be used (in the absence of significant high-energy gamma impurities such as Lu-174m and Ta-182).

Hf-175 radiography sources enable the use of the Small Controlled Area Radiography (SCAR) technique. Hf-175 radiography sources may be constructed with a smaller focal dimension than conventional sources, due to the high activation cross-section of Hf-174 and lower burn-away cross-section of Hf-175. The activation costs for Hf-175 may be substantially lower than those of Se-75, and activation of metal disks of Hf-174 to Hf-175 enables reliable source assembly techniques like those used to produce conventional Ir-192 radiography sources. Overall, disclosed Hf-175 gamma radiography sources provide advantageous, broad-use characteristics for gamma radiography in both the use context and manufacturing contexts.

Disclosed example gamma radiography sources include an encapsulated quantity of at least 10 Ci of Hafnium-175 (Hf-175).

In some example gamma radiography sources, the quantity of Hf-175 includes a plurality of stacked disks of at least one of metallic Hf-175, an alloy of Hf-175, or a compound containing Hf-175. In some example gamma radiography sources, the quantity of Hf-175 includes a plurality of stacked disks of at least one of a refractory material including Hf-175, a composite material including Hf-175, or a cermet including Hf-175. Some example gamma radiography sources further include a material which emits complementary gamma emissions to the quantity of Hf-175. In some example gamma radiography sources, the quantity of Hf-175 is formed as at least one of stacked disks, microspheres, pressed pellets, coiled wires, annuli, or profiled disks.

In some example gamma radiography sources, the quantity of Hafnium-175 is between 10-500 Ci. In some such examples, the quantity of Hafnium-175 is between 10-81 Ci. In some such examples, the quantity of Hafnium-175 is between 40-81 Ci. In some example gamma radiography sources, the quantity of Hf-175 is encapsulated in a welded metallic capsule.

Disclosed example gamma radiography sources include a quantity of at least 10 Ci of Hf-175 formed by neutron-irradiating, enriched Hf-174.

In some example gamma radiography sources, the quantity of Hf-174 includes a plurality of stacked disks of at least one of neutron-irradiated, enriched metallic Hf-174 or a neutron-irradiated, enriched alloy of Hf-174. In some example gamma radiography sources, the quantity of Hf-174 includes a plurality of stacked disks of at least one of a neutron-irradiated, enriched refractory material including Hf-174, a neutron-irradiated, enriched composite material including Hf-174, a neutron-irradiated, enriched cermet including Hf-174, or a neutron-irradiated, enriched compound including Hf-174.

Some example gamma radiography sources further includes a material which emits complementary gamma emissions to the quantity of neutron-irradiated, enriched Hf-174. In some example gamma radiography sources, the quantity of neutron-irradiated, enriched Hf-174 is formed as at least one of stacked disks, microspheres, pressed pellets, coiled wires, annuli, or profiled disks. In some example gamma radiography sources, the quantity of neutron-irradiated, enriched Hf-174 comprises at least one of stacked disks, microspheres, pressed pellets, coiled wires, annuli, or profiled disks, which are encapsulated in a sealed capsule.

In some example gamma radiography sources, the quantity of neutron-irradiated, enriched Hf-174 is enriched to at least 40% Hf-174, and Hf-180 in the gamma radiography source is less than 5%. In some example gamma radiography sources, the quantity of neutron-irradiated, enriched Hf-174 is enriched to at least 40% Hf-174, and Hf-180 in the gamma radiography source is less than 2%. In some example gamma radiography sources, the quantity of neutron-irradiated, enriched Hf-174 is enriched to at least 40% Hf-174, and Hf-180 in the gamma radiography source is less than 0.5%.

Disclosed example gamma radiation exposure devices include: a gamma radiation source comprising a quantity of Hf-175; and a shielding device configured to attenuate gamma radiation emitted by the gamma radiation source while the gamma radiation source is in a stored position, and configured to allow the gamma radiation source to be moved to an exposed position for gamma radiation exposure.

In some example gamma radiation exposure devices, the shielding device meets the requirements of a Type A package under the International Atomic Energy Agency Safety Standards, Regulations for the Safe Transport of Radioactive Material, 2012 Edition. Some example gamma radiation exposure devices further include a controller configured to be attached to the gamma radiation source and to control exposure of the gamma radiation source by controlling a position of the gamma radiation source with respect to the shielding device.

In some example gamma radiation exposure devices, the quantity of Hf-175 includes at least one of stacked disks, microspheres, pressed pellets, coiled wires, annuli, or profiled disks, which are encapsulated in a sealed capsule. In some example gamma radiation exposure devices, the gamma radiation source further comprises a material which emits complementary gamma emissions to the quantity of Hf-175.

Disclosed example methods to perform gamma radiography involve: positioning a gamma radiation source comprising a quantity of Hf-175 on a first side of a target object; positioning an imaging device on a second side of the target object opposite the gamma radiation source; and exposing the gamma radiation source to the target object to generate a radiographic image of the target object via the imaging device.

Disclosed example methods to manufacture a gamma radiation source involve irradiating a quantity of enriched Hf-174 using neutron irradiation, wherein the irradiation occurs in a location of a reactor which maximizes the combined thermal and epithermal flux relative to the fast neutron flux >5 MeV.

Some example methods further include forming the enriched Hf-174 into at least of one stacked disks, microspheres, pressed pellets, coiled wires, annuli, or profiled disks prior to the neutron irradiation. Some example methods further include forming the irradiated, enriched Hf-174 into at least one of one stacked disks, microspheres, pressed pellets, coiled wires, annuli, or profiled disks. Some example methods further include encapsulating the irradiated, enriched Hf-174 in a sealed capsule.

Disclosed example methods to manufacture a gamma radiation source involve irradiating a quantity of enriched Hf-174 using neutron irradiation, wherein the irradiation occurs in a location of a reactor wherein the epithermal flux is greater than 20% of the thermal flux.

Disclosed example methods to manufacture a gamma radiation source involve irradiating a quantity of enriched Hf-174 using neutron irradiation, wherein the irradiation occurs in a location of a reactor wherein the epithermal flux is greater than 30% of the thermal flux.

Disclosed example methods to manufacture a gamma radiation source involve irradiating a quantity of enriched Hf-174 using neutron irradiation, wherein the irradiation occurs in a location of a reactor wherein the epithermal flux is greater than 50% of the thermal flux.

Disclosed example methods to produce Hafnium-174-enriched Hafnium involve performing gaseous centrifugation of a compound containing Hafnium-174 and a single isotope of enriched Boron.

Disclosed example methods to produce Hafnium-174-enriched Hafnium involve performing atomic vapor laser isotope separation (AVLIS) of elemental Hafnium.

Disclosed example methods to produce Hafnium-174-enriched Hafnium involve performing molecular laser isotope separation (MLIS) of a compound containing Hafnium-174.

As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g.,” and “for example” set off lists of one or more non-limiting examples, instances, or illustrations.

While the present method and/or system has been described with reference to certain implementations, it will be understood by those skilled in the art that various changes may be made, and equivalents may be substituted without departing from the scope of the present method and/or system. For example, block and/or components of disclosed examples may be combined, divided, re-arranged, and/or otherwise modified. In addition, many modifications may be made to adapt a particular situation or material to the teachings of the present disclosure without departing from its scope. Therefore, the present method and/or system are not limited to the particular implementations disclosed. Instead, the present method and/or system will include all implementations falling within the scope of the appended claims, both literally and under the doctrine of equivalents.

1. A gamma radiography source comprising an encapsulated quantity of at least 10 Ci of Hafnium-175 (Hf-175).

2. The gamma radiography source as defined in claim 1, wherein the quantity of Hf-175 comprises a plurality of stacked disks of at least one of metallic Hf-175, an alloy of Hf-175, or a compound containing Hf-175.

3. The gamma radiography source as defined in claim 1, wherein the quantity of Hf-175 comprises a plurality of stacked disks of at least one of a refractory material including Hf-175, a composite material including Hf-175, or a cermet including Hf-175.

4. The gamma radiography source as defined in claim 1, further comprising a material which emits complementary gamma emissions to the quantity of Hf-175.

5. The gamma radiography source as defined in claim 1, wherein the quantity of Hf-175 is formed as at least one of stacked disks, microspheres, pressed pellets, coiled wires, annuli, or profiled disks.

6. The gamma radiography source as defined in claim 1, wherein the quantity of Hafnium-175 is between 10-500 Ci.

7. The gamma radiography source as defined in claim 6, wherein the quantity of Hafnium-175 is between 10-81 Ci.

8. The gamma radiography source as defined in claim 7, wherein the quantity of Hafnium-175 is between 40-81 Ci.

9. The gamma radiography source as defined in claim 1, wherein the quantity of Hf-175 is encapsulated in a welded metallic capsule.

10. A gamma radiography source comprising a quantity of at least 10 Ci of Hf-175 formed by neutron-irradiating enriched Hafnium-174 (Hf-174).

11. The gamma radiography source as defined in claim 10, wherein the quantity of Hf-174 comprises a plurality of stacked disks of at least one of neutron-irradiated, enriched metallic Hf-174 or a neutron-irradiated, enriched alloy of Hf-174.

12. The gamma radiography source as defined in claim 10, wherein the quantity of Hf-174 comprises a plurality of stacked disks of at least one of a neutron-irradiated, enriched refractory material including Hf-174, a neutron-irradiated, enriched composite material including Hf-174, a neutron-irradiated, enriched cermet including Hf-174, or a neutron-irradiated, enriched compound including Hf-174.

13. The gamma radiography source as defined in claim 10, further comprising a material which emits complementary gamma emissions to the quantity of neutron-irradiated, enriched Hf-174.

14. The gamma radiography source as defined in claim 10, wherein the quantity of neutron-irradiated, enriched Hf-174 is formed as at least one of stacked disks, microspheres, pressed pellets, coiled wires, annuli, or profiled disks.

15. The gamma radiography source as defined in claim 10, wherein the quantity of neutron-irradiated, enriched Hf-174 comprises at least one of stacked disks, microspheres, pressed pellets, coiled wires, annuli, or profiled disks, which are encapsulated in a sealed capsule.

16. The gamma radiography source as defined in claim 10, wherein the quantity of neutron-irradiated, enriched Hf-174 is enriched to at least 40% Hf-174, and Hf-180 in the gamma radiography source is less than 5%.

17. The gamma radiography source as defined in claim 10, wherein the quantity of neutron-irradiated, enriched Hf-174 is enriched to at least 40% Hf-174, and Hf-180 in the gamma radiography source is less than 2%.

18. The gamma radiography source as defined in claim 10, wherein the quantity of neutron-irradiated, enriched Hf-174 is enriched to at least 40% Hf-174, and Hf-180 in the gamma radiography source is less than 0.5%.

19. A gamma radiation exposure device, comprising:
a gamma radiation source comprising a quantity of Hf-175; and
a shielding device configured to attenuate gamma radiation emitted by the gamma radiation source while the gamma radiation source is in a stored position, and configured to allow the gamma radiation source to be moved to an exposed position for gamma radiation exposure.

20. The gamma radiation exposure device as defined in claim 19, wherein the shielding device meets the requirements of a Type A package under the International Atomic Energy Agency Safety Standards, Regulations for the Safe Transport of Radioactive Material, 2012 Edition.

21-34. (canceled)
